(19) United States
(12) Patent Application Publication
(10) Pub. No.: US 2011/0124975 A1
(43) Pub. Date: May 26, 2011

(54) Method for Medical Diagnosis Utilizing PDA Software Robots

(76) Inventor: Arthur Solomon Thompson, Reston, VA (US)

(21) Appl. No.: 13/020,969

(22) Filed: Feb. 4, 2011

Related U.S. Application Data
(63) Continuation-in-part of application No. 11/723,138, filed on Mar. 16, 2007, now abandoned.

(51) Int. Cl. A61B 5/00

(52) U.S. Cl. 600/300

(57) ABSTRACT

A method for diagnosing problems using piece removal techniques and which can include establishing a health status variable, collecting and storing information from a user in the health status variable tracking the health status variable, comparing the health status variable to a knowledge base, using the health status variable to reduce the size of the knowledge base, leveraging patterns and past history to formulate a search strategy, using the search strategy to collect further diagnostic information from the user, updating the health status variable, comparing the updated health status variable to the reduced knowledge base, diagnosing a problem based on the further diagnostic information, and selecting treatment for the problem.

### CROSS REFERENCE TO RELATED APPLICATIONS

This patent application is a continuation of pending U.S. patent application Ser. No. 11/723,138, filed Mar. 16, 2007 and U.S. patent application Ser. No. 12/232,335, filed on Sep. 16, 2008, the contents of which are incorporated by reference in their entirety herein.

### BACKGROUND

Determining a medical diagnosis is often difficult. Many factors must be accounted for, including past medical history and current symptoms. Often similar symptoms can result from vastly different medical causes. In addition, symptoms can interact with each other in unexpected ways, and the relationship between current symptoms and symptoms that have occurred in the past can be deceptive and confusing.

The art of deciphering medical symptoms is esoteric and inaccessible to most. This makes practicing preventive medicine, which could reduce healthcare costs a great deal, intimidating for those with little medical training. It is often difficult to determine when a combination of symptoms is harmless and can be safely ignored, and when the advice of a physician should be sought.

### SUMMARY

In one exemplary embodiment, a method for diagnosing problems using piece removal techniques may be described. The method can include establishing a health status variable; collecting information from a user; storing the information in the health status variable; tracking the state health status variable to determine patterns and past history; comparing the health status variable to a knowledge base containing other health status variables; reducing the size of the knowledge base using that comparison; leveraging patterns and past history associated with the health status variables remaining in the knowledge base to formulate a search strategy; collecting further diagnostic information from the user consistent with the search strategy; updating the health status variable; comparing the updated health status variable to the reduced knowledge base; diagnosing a problem based on the further diagnostic information; and selecting treatment.

In another exemplary embodiment, a method for diagnosing problems using equilibrium-reestablishing techniques can be described. The method can include establishing a health status variable; collecting information from a user; storing the information from the user in the health status variable; tracking the state of health status variable to determine patterns and past history; identifying differences between previous versions of the user's health status variable and the user's current health status variable; collecting further diagnostic information from the user regarding those differences; updating the health status variable associated with the user; comparing the updated health status variable to previous versions of the user's health status variable; diagnosing a problem based on the further diagnostic information; and selecting treatment for the problem.

### BRIEF DESCRIPTION OF THE DRAWINGS

Embodiments of the invention will now be further described with reference to the accompanying drawing.

FIG. 1 is a diagram of an exemplary embodiment of a method for medical diagnosis.

FIG. 2 is an exemplary diagram showing the leveraging of data in a medical diagnostic system.

FIG. 2a is an exemplary diagram showing an electronic medical record with a medical diagnosis advisory.

FIG. 3 is an exemplary diagram showing a scheduling agent.

FIG. 4 is an exemplary diagram showing a piano model of a diagnosis system.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance, or illustration.” Any embodiment described herein as “exemplary” is not necessarily to be construed as preferred or advantageous over other embodiments. Likewise, the term “embodiments of the invention” does not require that all embodiments of the invention include the discussed feature, advantage or mode of operation.

Further, many embodiments are described in terms of sequences of actions to be performed by, for example, elements of a digital control system and the digital signal processing (DSP) devices. It will be recognized that various actions described herein can be performed by specific circuits (e.g., application specific integrated circuits (ASICs)), by program instructions being executed by one or more processors, or by a combination of both. Additionally, these sequences of actions and processes described herein can be considered to be embodied entirely within any form of computer platform having stored therein a corresponding set of computer instructions that upon execution would cause an associated processor to perform the functionality described herein or otherwise communicate, for example through a network, with any other computing devices to perform a desired functionality. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

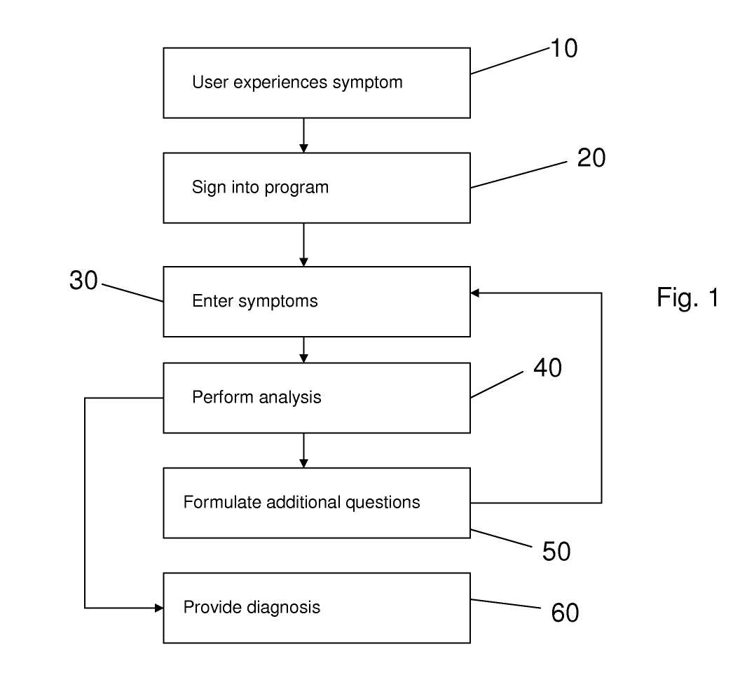

FIG. 1 shows an exemplary diagram of a process for considering current medical symptoms in combination with past medical history in order to provide a diagnosis. At step 10, a user can experience symptoms from an unknown medical condition. At step 20, the user can access a device running the diagnostic system. The device can then access and display the medical history, if any, that is associated with the user. At step 30, the user can access a body component menu to select body components and associated symptoms which the user is experiencing. The system can use these selections to modify a health status variable associated with the user. At step 40, the system can perform an analysis of the medical history and current health status variable associated with the user. At step 50 the system can use this analysis to identify other body components and symptoms potentially related to the selections made by the user. These potential related body components and symptoms can be identified by patterns emerging in the number of times a particular body component has exhibited a particular symptom, the number of times a particular symptom has arisen in the medical history, or by any other means. The system can then return to step 30 to suggest these potential related body components or symptoms to the user and seek additional input. Alternatively, at step 40 the system can determine that the analysis is sufficient to provide a diagnosis. The system can then move to step 60 and present the diagnosis to the user and update the medical history of the user with the current health status variable.

The health status variable described in step 30 can take the form of a binary string of any length, for instance two hundred bits. Each bit in the health status variable binary string can represent a body component, for instance blood, urine, vital sign, or any other desired component. The state of each bit can indicate that the body component related to the bit is healthy/unhealthy, acceptable/unacceptable, pass/fail, or any other condition. At step 30, when a user identifies a body component that is exhibiting a symptom, the bit in the health status variable corresponding to that body component can be changed from one state to the other, for instance from healthy to unhealthy.

The analysis in step 40 can take place in a variety of ways. In one exemplary embodiment, this analysis can take the form of a search-driven, piece-removal or process-of-elimination heuristic. The system can attempt to make a diagnosis by comparing the health status variable of the user with other health status variables stored in a knowledge base. When the patient provides symptoms to the system, the size of the knowledge base that must be covered in order to make a diagnosis can be narrowed by eliminating stored health status variables that are inconsistent with the provided symptom. On subsequent iterations of the loop of steps 30 through 50, the system can interrogate the patient with questions and suggestions for related body components, and can then further prune down the knowledge base. Adding clues, detecting symptoms, or answering questions can establish constraints on the knowledge base. The knowledge base can be further narrowed by leveraging the medical history associated with the user, which can include past instantiations of the health status variable of the user, along with a count of the number of times that a body component has been indicated or a symptom has appeared in the user's health status variable.

In another exemplary embodiment, the analysis performed in step 40 can take the form of a corrective, equilibrium-reestablishing heuristic. The system can compare the user's current health status variable to previous versions of the user's health status variable, under the assumption that the previous versions represented an equilibrium, or healthy state of the user. The system can then collect all of the aberrations and complete the diagnosis by identifying the set of objects which needs to be restored to return the health status variable of the user to its previous state.

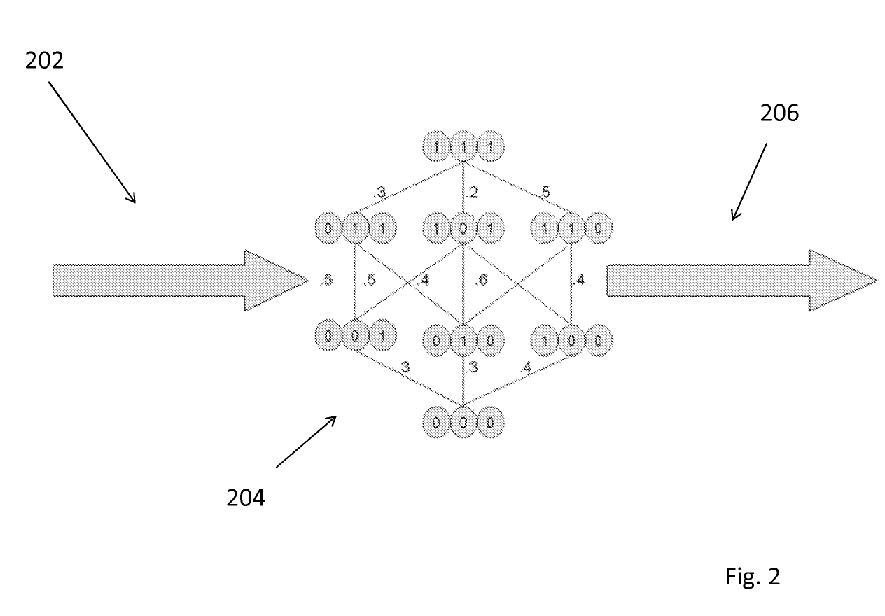

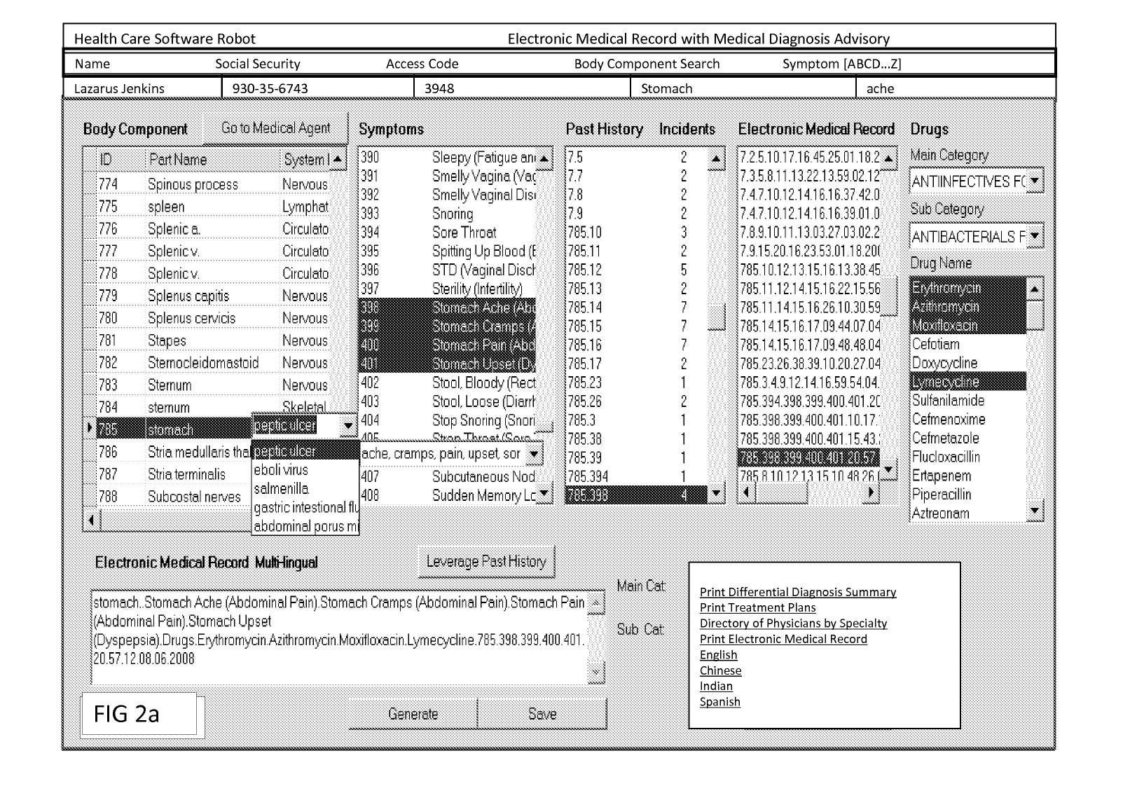

In still a further exemplary embodiment, the analysis performed in step 40 can be frequency-based. Frequency information can be collected from the frequency that the symptom or body component appears in the user's medical history. This information can be leveraged to develop a frequency-driven input strategy to alleviate symptoms using a frequency treatment such as entrainment. The body can be viewed as a frequency spectrum considering body cells and body components, allowing a frequency treatment approach to restoring full frequency oscillation harmony to be adopted. Symptoms can form frequency signatures which can be pattern-matched with patterns that have arisen in the search history. This frequency-recognition model can allow the processing of strings of very long length. This can be seen as analogous to a piano, the fundamental design of which allows for playing millions of songs on just 88 basis keys. This process can proceed as illustrated in exemplary FIG. 2. At 202, information is supplied to the system, at 204 the frequency-based search and decision-making heuristic is applied to the information provided, and at 206 a pattern-matched result is provided that can be used to further refine the search or provide a diagnosis.

Referring now to exemplary FIG. 2a, data compression or mapping can provide for software robot capabilities on mobile electronic devices, such as, but not limited to PDAs and cell phones. Exemplary FIG. 2a shows an interface enumerating many software robots capability, however specifically the field labeled “Past History and Incidents” can capture exemplary embodiments such as the piano algorithm that may be a collection of basis vectors and their commensurate visit counts. By reusing basis vector combinations or systems great data storage savings can be realized promoting an objective of software robots.

Additionally, and referring back to exemplary FIG. 2, a search strategy that leverages past history in a matter of seconds may be utilized. In this exemplary embodiment, of software robot search pattern matching (i.e. like patterns) of input collection on graph frequency based topology can provide for the capture of an entire state past history of three (or N) bits. Virtually any strings past history can be identified in seconds also exploiting a virtually constant data foot print based on a finite set of basis vectors spanning the problem space.

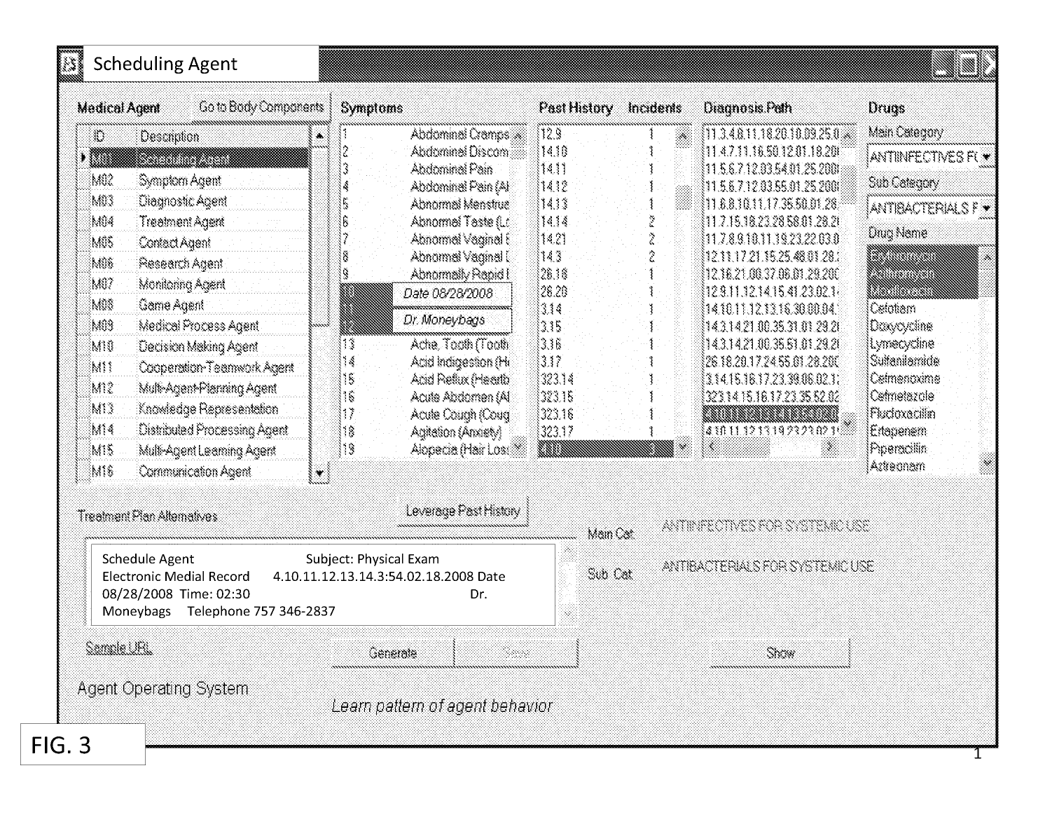

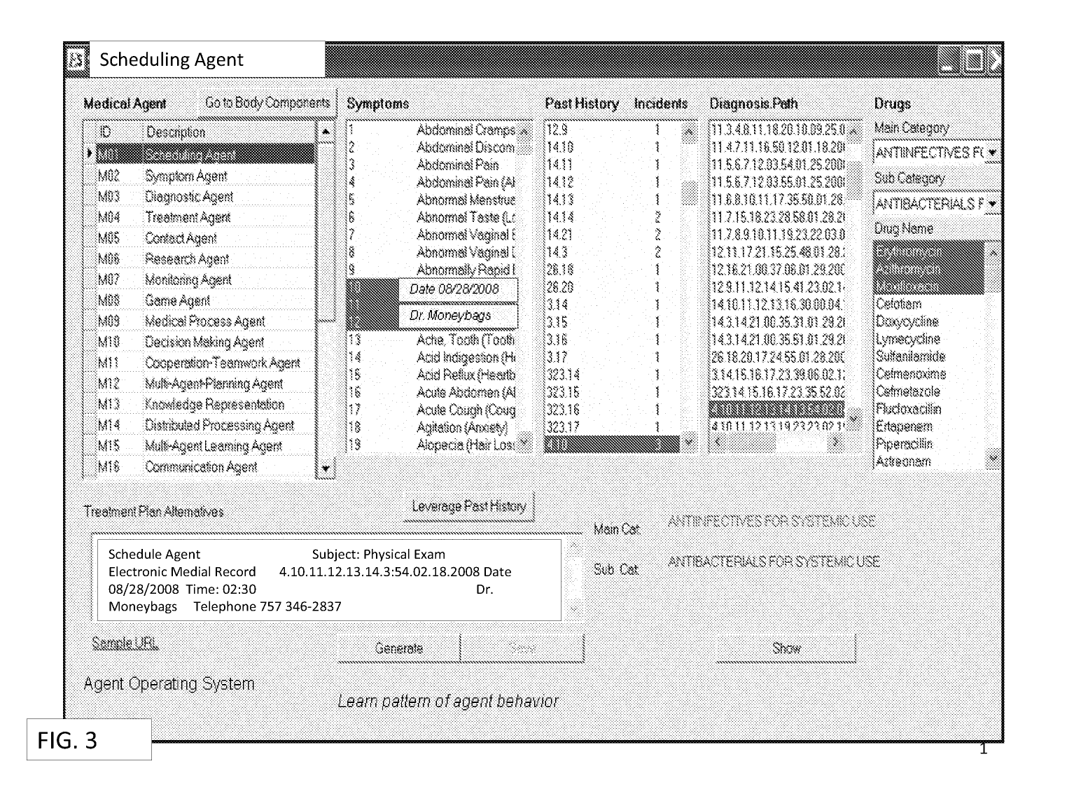

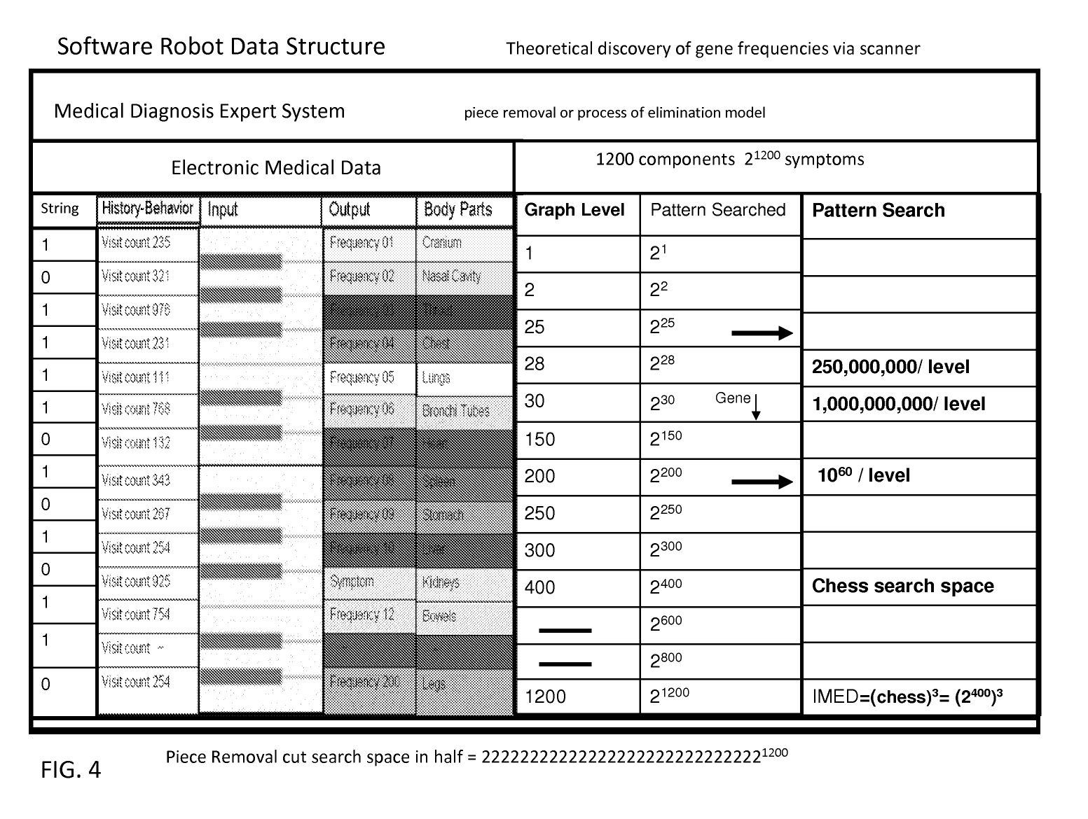

In still another exemplary embodiment, and referring now to FIGS. 3 and 4, other aspects of embodiments may be elaborated upon. Here, an agent can augment the capability, scalability and scope of applications defined on a software robot. Information storage (such as piano model storage, as described above) can allow for storage of any size string and information retrieval can leverage any past history nearly instantaneously. Additionally, an application can use a software robot's GUI design for a PDA or any other desired telecommunications interface. This can further allow for an electronic medical data application with an interface that may provide for the input, to a medical diagnostic application, such as specified as a field's body component. Further, in any of the exemplary embodiments, any language used or found in history data may be translated as desired and text in any format may be read, interpreted and leveraged as desired. Additionally, in exemplary embodiments, the data can be interpreted as showing a piano-like frequency recognition of strings having thousands of bits. Further, with reference to exemplary FIG. 3, software robots can use agents to extend the capability of existing software robots and libraries of agents can be used to perform simple to complex tasks that may facilitate information assurance objectives.

In still further exemplary embodiments, a method of establishing optimized processes for intelligent search can be realized using an expert domain of basis vectors or objects. The basis vectors or objects can have a linear combination that spans a vector space. As discussed previously, such a combination can resemble a piano, for example have eighty eight keys. The vectors can then be mapped to any number of body components, for example 200 to 1000 body components. This number can include a list of objects that can be used to select various body symptoms or aberrations. Further, as these components or vectors undergo system cycles, visit counts can be recorded to define the propensity of a component to be visited based on prior logging of medical conditions. Thus, as the frequency reading of the symptom components are recorded, the commensurate frequency reading of the body components can be recorded for further processing and diagnosis may be provided after the system questions and symptoms are documented.

Further, and referring to exemplary FIG. 2, another exemplary searching and diagnosis methodology and system may be described. Here, the search can be an optimized intelligent search that utilized pattern reduction of the search domain through piece removal, search heuristic using piece removal and graph traversal strategy. This search can then leverage any amount of past history using a basis set of vectors spanning the problem domain and applying a visit count to basis systems to capture past history. Thus, a graph heuristic to optimize the processing of a collection of objects can be generated and utilized. Further, the accessing of medical history rapidly, for example in a matter of seconds, can be realized through the use of a finite basis set. The finite basis set can capture all states of a system in addition to recording a system visit count. This can then provide the necessary routing of past medical history to new medical conditions.

In another exemplary embodiment, a method of facilitating the capabilities of a PDA software robot for internalizing information as IMED architecture selecting body components and symptoms mapped to numbers that can be used as pattern matched strings of any desired amount of past medical history in a frequency domain can be described. The methodology may further find treatment plans from prior solutions, for example, from a large frequency storage interface as a general system problem solver software robot leverages a basis set of objects that can span a vector space. A frequency-base signature can then be realized by formulating and concatenating objects. The signature may then be pattern matched with historic patterns to identify solutions to any desired problem. Further, such architecture can support the learning of any number of process and procedures can have a multi-lingual communications expert shell for support of any desired number of decision and problem analysis applications.

In still a further exemplary embodiment, a language robot can leverage any available history frequency signatures in order to establish context and meaning of word phrases. Such a system may be designed to capture all spoken language, in order to compare and leverage all past phrases. Thus, past medical diagnoses including common words or phrases may be intelligently interpreted so as to provide for accurate and near instantaneous results.

In another exemplary embodiment, PDA software robots can leverage past history. As described herein, any number of factors may be evaluated through the entry of data and the evaluation of any available historical data. Examples of various implementations that may be utilized includes, but is not limited to: Real-Time (Embedded) Self Healing Networks, Control Systems Vulnerability Analysis, Operating Systems (OS & IOS) Engineering Control Systems, Protocols Engineering and Applied Sciences, Legacy Networked Applications Process and Data Modeling, Unprecedented Networked Applications Chemical Controls Systems, Sensor-Based Mechanical Controls Systems, Location-Based Electrical Controls Systems, Grid-Based Expert Systems, Swarm-Based Decision Support System, Known” Non-Networked Applications, Business Models (data flow, optimization), Summary of IPV6 Search Engines Financial Models (cash flow, break even), Target IPV6 Intelligent Brokers Search Engines, Health Care (diagnosis, analysis, treatment) Diagnostic agent, Agent Reading and Comprehension Systems, Trouble shooting agent exceptional Treatment Plan agent, Security Initiatives (i.e. leveraging past history of Medical Record-History agent defense vehicles), Medical Scenario and Games agent (Machine Games learning), Recreation (children, adult) (prototyped evaluation), Medical Process and Operating Room Procedures function), Agent Educational (children, adult), Business Control Systems Simulation/Tactical, Strategic Business Development Models, Computer Animation Finance Model Mathematics and Applied Sciences, Loan Development Models (prototyped), Fourier analysis Enterprise Resource Planning, Set Theory and Deployment Business, Data Flow Models Solution of Turing compliant Strings, Operating Systems, General System Problem Solver Software Development, Non linear to Linear Transform Integrated Development Environment, N dimensional Problem Solver Advance Software Modeling and Deployment Unprecedented Network, Applications Software (networks, operating system, application, Grid-Based-Navigation for Autonomous, Mobile software) Robots Hardware (configuration, performance), Swarm Based-Software Architecture for Radio, Real-time (embedded) Based Mobile Self-Organizing Systems, Trouble Shooting Systems (prototyped), Sensor Based-network designed run on Software Robotics Bluetooth using the Serial Port Profile, Intelligent Agents Location Based-software on the smart phone and Defense and Tactical Systems IPV6 and OIS.

In addition, with each diagnosis the system can work to optimize its efficiency. Each episode can be recorded by the system as a collection of health status variables which span the relevant problem space. These collections can be stored, and can be compared with any new inputs, so that when an identical series of inputs occurs the previous diagnosis can be retrieved.

Further, the exemplary system herein can be implemented on any device, for example as a program running on a computer, smartphone, or digital handheld apparatus, personal digital assistant (PDA) or on a web server and presented as a website. Additionally, any known form of interface may be utilized for the input or display of data and information may be shared or communicated across any form of wired or wireless transmission.

The foregoing description and accompanying drawings illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method for diagnosing problems using piece removal techniques, comprising:
establishing a health status variable, the health status variable containing a plurality of elements corresponding to body components;
collecting information from a user regarding the status of the user's body components;
storing the information from the user by altering the state of the corresponding elements in the health status variable;
tracking the state of the plurality of elements in the health status variable to determine patterns and past history;
comparing the health status variable to a knowledge base containing other health status variables;
reducing the size of the knowledge base by removing health status variables and objects based on the collected information, patterns, and past history;
leveraging patterns and past history associated with the health status variables remaining in the knowledge base to formulate a present search strategy;
collecting further diagnostic information from the user consistent with the present search strategy;
updating the health status variable associated with the user by altering the state of the corresponding elements in the health status variable;
comparing the updated health status variable to the reduced knowledge base;
diagnosing a problem based on the further diagnostic information; and
selecting treatment for the problem based on the further diagnostic information, patterns, and past history

2. The method of claim 1, wherein the health status variable is a binary string.

3. The method of claim 2, wherein the binary string contains approximately 200 bits.

4. The method of claim 2, wherein each bit of the binary string corresponds to a body component of the user.

5. The method of claim 4, wherein collecting information from the user further comprises changing the bit in the health status variable corresponding to each body component in a way that reflects the state of that body component.

6. The method of claim 1, further comprising storing the health status variables associated with each diagnosis in order to optimize subsequent diagnoses of the same type.

7. A method for diagnosing problems using equilibrium-reestablishing techniques, comprising:
establishing a health status variable, the health status variable containing a plurality of elements corresponding to body components;
collecting information from a user regarding the status of the user's body components;
storing the information from the user by altering the state of the corresponding elements in the health status variable;
tracking the state of the plurality of elements in the health status variable to determine patterns and past history;
identifying differences between previous versions of the user's health status variable and the user's current health status variable;
collecting further diagnostic information from the user regarding those differences;
updating the health status variable associated with the user by altering the state of the corresponding elements in the health status variable;
comparing the updated health status variable to previous versions of the user's health status variable;
diagnosing a problem based on the further diagnostic information; and
selecting treatment for the problem based on the further diagnostic information, patterns, and past history.

8. The method of claim 7, wherein the health status variable is a binary string.

9. The method of claim 8, wherein the binary string contains approximately 200 bits.

10. The method of claim 8, wherein each bit of the binary string corresponds to a body component of the user.

11. The method of claim 10, wherein collecting information from the user further comprises changing the bit in the health status variable corresponding to each body component in a way that reflects the state of that body component.

12. The method of claim 7, further comprising storing the health status variables associated with each diagnosis in order to optimize subsequent diagnoses of the same type.

13. A system for diagnosing problems using comprising:
an apparatus having a processor and a display and data entry device;
an interface for prompting and entering information;
a knowledge base housing information; and
a computer program process that establishes a health status variable, collects and stores information from a user in the health status variable, tracks the health status variable, compares the health status variable to a knowledge base, uses the health status variable to reduce the amount of the knowledge base that needs to be searched, leverages patterns and past history to formulate a search strategy, uses the search strategy to collect further diagnostic information from the user, updates the health status variable, compares the updated health status variable to the reduced knowledge base, diagnoses a problem based on the further diagnostic information, and selects treatment for the problem.
